(19) United States
(12) Patent Application Publication
(10) Pub. No.: US 2004/0132005 A1
(43) Pub. Date: Jul. 8, 2004

(54) Method of forming biochip and application thereof

(76) Inventors: Jing-Hsiang Hsu, Keelung City (TW); Wen-Yih Chen, Jungli City (TW); Rong-Seng Chang, Taipei City (TW); Rong-Nan Huang, Jhongli City (TW); Wen-Ren Li, Pingjhen City (TW)

Correspondence Address: J.C. Patents, SUITE 250, 4 VENTURE, IRVINE, CA, 92618, US

(21) Appl. No.: 10/660,139

(22) Filed: Sep. 10, 2003

(30) Foreign Application Priority Data
Sep. 10, 2002 (TW) 91120544

(51) Int. Cl. C12Q 1/00

(52) U.S. Cl. 435/4

(57) ABSTRACT

A method of forming a biochip in which a micro-carrier having a bar code or a number code thereon is provided. A surface-modifying step is performed for modifying a surface of the micro-carrier to an aminated surface. A solid-phase peptide synthesis step is then conducted to synthesize a peptide having a specific amino acid sequence on the aminated surface of the micro-carrier or immobilizing an antibody or an antigen on the aminated surface of the micro-carrier. The micro-carrier can be applied on a biological activity analysis or an antibody-antigen interaction analysis, and the results can be read out directly from the bar code or the number code on the micro-carrier.

### CROSS-REFERENCE TO RELATED APPLICATION

This application claims the priority benefit of Taiwan application serial no. 91120544, filed on Sep. 10, 2002.

### BACKGROUNDING OF THE INVENTION

1. Field of Invention

The present invention relates to a fabrication method for a biochip and an application method thereof. More particularly, the present invention relates to a method for fabricating a biochip for a rapid detection of the biological activity of a peptide and the antigen-antibody interaction.

2. Description of Related Art

The discovery and development of new drugs, especially peptide and protein types of drugs, is the most important topic in the development of biotechnology. Conventionally, the design and the development of new drugs rely on the application of different synthetic chemistry techniques to synthesize a vast amount of chemical substances of various structures, and the detection of the biological activities of these chemical substances. However, the detection of the biological activities of these chemical substances requires the application of tedious and intricate experiments of the cell and tissue culture techniques.

The understanding of the specific recognition and binding activities of biological compounds and the biological activity of a chemical material can be predicted by the binding abilities of certain biological materials (e.g. membrane protein). Especially, the application of biochemical materials is the mainstream approach in the recent development of drugs (e.g. peptide and protein type of drugs). At least in the design of a lead compound, biochemical materials are often used in the development of drugs. Further, since the methods for synthesizing a peptide are more sophisticated, specific and nonspecific peptide synthesis can be achieved rapidly using synthetic chemistry.

Moreover, the detection method for pathogens of many current diseases relies on the particular and specific binding characteristics of antigen-antibody. However, the conventional antigen-antibody detection method requires a series of reactions between a reagent and a test sample. The conventional detection method is thus very time-consuming.

### SUMMARY OF THE INVENTION

Accordingly, the present invention provides a fabrication method and an application of a biochip, wherein a speedy analytical method for the design of a new drug is provided.

In accordance to the present invention of a fabrication method and an application of a biochip, the disadvantages of being time-consuming and labor intensive in detecting the biological activity and the antibody-antigen interaction are obviated.

The present invention can further provide a fabrication method and an application of a biochip, wherein a speedy, convenient and easily operated detection method is developed.

The present invention provides a fabrication method for a biochip, wherein this method includes providing a micro-carrier. Further, the micro-carrier is already labeled with a bar code or a number code. A material in forming the micro-carrier includes polyethylene terephthalate (PET). The labeling of the micro-carrier with either a bar code or a number code can refer to U.S. Pat. No. 6,350,620. Thereafter, a surface modification step is performed to modify the surface property of the micro-carrier to an aminated surface. In the present invention, the surface modification procedure comprises covering a silicon dioxide layer on the surface of the micro-carrier, followed by reacting the silicon dioxide layer with 3-aminopropyltriethoxysilane to modify the surface of the micro-carrier to an aminated surface. Thereafter, a solid-phase peptide synthesis step is conducted to synthesize a peptide with a specific amino acid sequence on the aminated surface.

Using the biochip formed according to the above method, the peptide with a specific amino acid sequence and the specific bar code (number code) establish coordinated information on a peptide and a bar code (or number code), which can be used on the research and development of new peptide drugs. Moreover, the microchip that comprises with a peptide of a specific amino acid sequence can be applied to the detection of a specific biological molecule.

The present invention provides a fabrication method for a biochip, wherein this method includes providing a micro-carrier, and this micro-carrier is already labeled with a bar code or a number code. The micro-carrier is formed with a material that includes, for example, polyethylene terephthalate (PET). The method for labeling the micro-carrier with a bar code or a number code can be referred to the U.S. Pat. No. 6,350,620. Thereafter, a surface modification step is performed to modify the surface of the micro-carrier to an aminated surface. In the present invention, the surface modification step includes covering the surface of the micro-carrier with a layer of silicon dixoide, followed by reacting the silicon dioxide layer with 3-aminopropyltriethoxysilane to modify the surface of the micro-carrier to an aminated surface. Thereafter, an antigen or antibody is immobilized on the aminated surface of the micro-carrier. The biochip formed according to the above method can be applied to the detection of an antigen-antibody interaction.

According to the fabrication method and the application of a biochip of the present invention, the micro-carrier is labeled with a bar code or a number code. After using the micro-carrier to detect the biological activity or the interaction between an antigen-antibody, only an optical apparatus is required to directly read the bar code or number code, and the sequence of a biological molecule or a test-pending material that corresponds to the antibody (antigen) is confirmed.

The fabrication method and the application of a biochip of the present invention further provides a speedy method for detecting the biological activity of a peptide and the interaction of an antigen-antibody. The disadvantages of being time-consuming and labor intensive of the prior art approaches are resolved.

The fabrication method and the application of a biochip of the present invention can be applied directly on biological tissues for biological detection.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. The patent or application file contains at least one drawing executed in color. Copies of this patent or patent application publication with color drawing(s) will be provided by the Office upon request and payment of the necessary fee.

FIG. 1 is a flow chart of steps illustrating the fabrication method and the application of a biochip according to one aspect of the present invention.

FIG. 2 is a picture of a biochip labeled with a number code according to one aspect of the present invention.

FIG. 3 is a schematic diagram illustrating the chemical reaction for a micro-carrier surface modification step according to one aspect of the present invention.

FIG. 4 is a picture of a test result for confirming whether the surface of the micro-carrier has been modified to an aminated surface.

FIG. 5 is a picture of a test result for confirming whether the amine group of the amino acid on the micro-carrier is exposed.

FIG. 6 a schematic diagram illustrating the detection of biological activity according to one aspect on the present invention.

FIG. 7 is a flow chart of steps illustrating the fabrication method and the application of a biochip according to another aspect of the present invention.

FIG. 8 is a schematic diagram illustrating the detection of an antibody-antigen interaction according to another aspect of the present invention.

### DESCRIPTION OF THE PREFERRED EMBODIMENTS

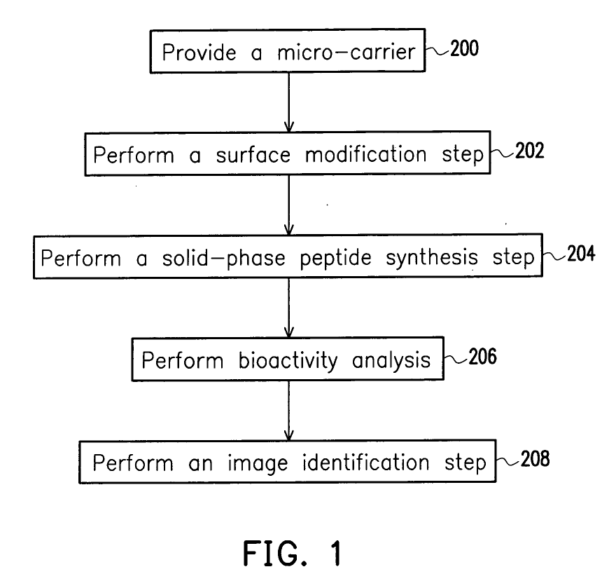

FIG. 1 is a flow diagram of steps, illustrating the fabrication method for a biochip and the application thereof according to one aspect of the present invention.

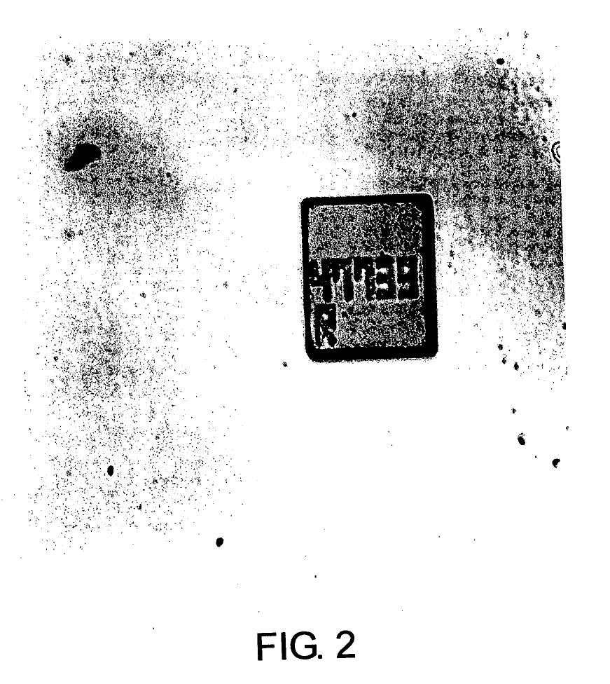

Referring to FIG. 1, a micro-carrier is provided (step 200), wherein the micro-carrier is labeled with an identification code (for example, a bar code or a number code). As shown in FIG. 2, FIG. 2 illustrates a micro-carrier with a dimension of 100 micron×100 micron. The micro-carrier is labeled with a group of numbers. In this aspect of the present invention, the micro-carrier is a high molecular weight material. This high molecular weight material is, for example, polyethylene terephthalate (PET).

Referring back to FIG. 1, a surface modification step (step 202) is conducted. In this aspect of the invention, the surface modification step comprises coating a silicon dioxide layer on the surface of the micro-carrier. The silicon dioxide layer surface is further modified to an aminated surface. The modification step is detailed as follow.

1. A PET micro-carrier coated with a silicon dioxide layer is soaked in isopropyl alcohol and is cleaned for 25 minutes in an ultrasonic bath.

2. The isopropyl alcohol is removed and is replaced by methanol. The PET micro-carrier soaked in methanol is further ultrasonically cleaned for another 25 minutes.

3. The PET micro-carrier is removed from the ultrasonic bath and is blown dry using a nitrogen gas.

4. The PET micro-carrier is then placed in a cleaning solution (a mixture of 5 ml of 31% H₂O₂ and 5 ml of 72 M H₂SO₄) and is ultrasonically cleaned for 6 hours.

5. The carrier is further removed from the ultrasonic bath and is rinsed with a large amount of deionized water, followed by blow-drying with a nitrogen gas.

6. The PET carrier is again soaked in methanol and is ultrasonically cleaned for 5 minutes.

The above steps 1 to 6 are for cleaning the micro-carrier to expose the hydroxyl group (OH—) on the silicon dioxide layer surface. The micro-carrier can remain submerged in methanol for preservation if the micro-carrier is not being used imminently.

Thereafter, the silicon dioxide surface of the micro-carrier is modified to an aminated surface. The modification procedure is detailed as follow.

7. The micro-carrier is blown-dry after being removed from methanol.

8. The micro-carrier is placed in a test tube and is vacuum dried. The test tube is filled with an argon gas.

9. 2 ml of 3-aminopropyltriethoxysilane and 8 ml of the 99.5% ethanol are injected into the test tube, followed by shaking the test tube for 6 hours.

10. The micro-carrier is rinsed with methanol for several times and is then vacuum dried subsequently.

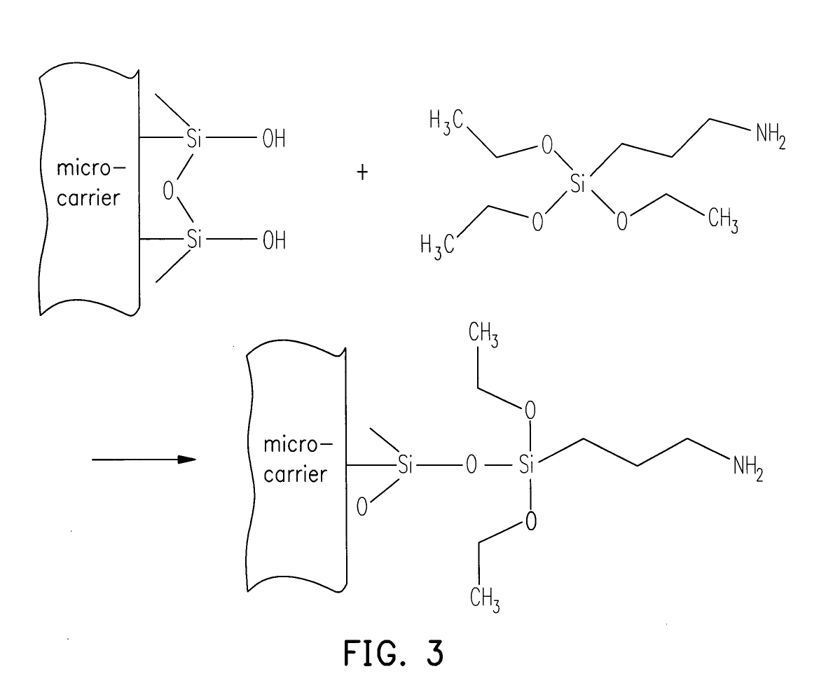

After completing the above process steps, the silicon dioxide surface of the micro-carrier is converted to an animated surface. As shown by the chemical reaction of the surface modification step in FIG. 3, the hydroxyl groups (OH—) on the silicon dioxide layer surface of the micro-carrier are exposed. After reacting with 3-aminopropyltriethoxysilane, the silicon dioxide layer surface is converted to an aminated surface.

To confirm the surface of the micro-carrier has been converted to an aminated surface, a test is conducted. The test is based on the Ninhydrin assay, which is detailed as follow. 10 gm of phenol is added to 2.5 ml of ethanol to form solution (1). 16.25 mg of potassium cyanide is dissolved in 25 ml of water. 0.5 ml of the potassium cyanide solution is diluted to 50 ml of solution (2) using pyridine. Solution (1) is mixed with solution (2) to form solution (A). 2.5 gm of Ninhydrin is dissolved in 50 ml of the 99.5% ethanol to form solution (B). The micro-carrier is then soaked in a mixture solution of solution (A) and solution (B). The color change of the mixture solution is observed.

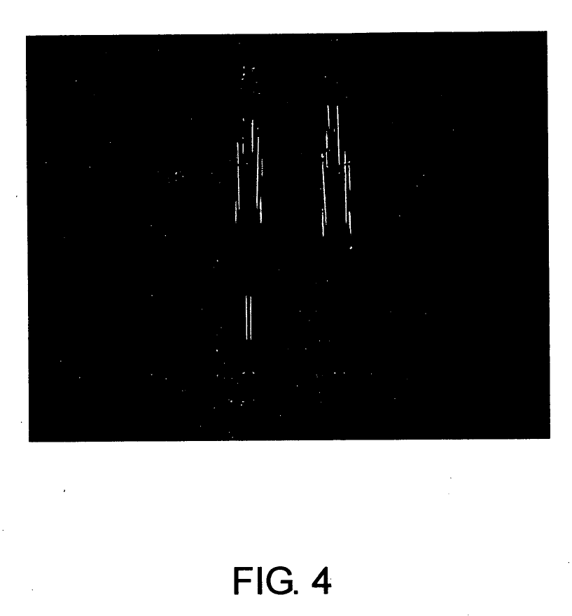

Referring to FIG. 4, a micro-carrier is placed inside test tube 500, wherein the surface of the micro-carrier 500 is covered with a silicon dioxide layer. Another micro-carrier is placed inside test tube 502, wherein the surface of this micro-carrier has been modified to an animated surface. 400 μl of solution (A) and 100 μl of solution (B) are added to test tube 500 and test tube 502, respectively. The test tubes 500, 502 are heated to 100 degrees Celsius. Since the Ninhydrin assay is used, the solution would exhibit a violet color if the amine groups were present, whereas the solution would exhibit a yellow color if the amine groups were absent. As shown in FIG. 4, the solution in test tube 500 is yellow in color, while the solution in test tube 502 is violet in color. Accordingly, the surface of the micro-carrier in test tube 502 (the micro-carrier after being subjected to the surface modification process step has been modified to an aminated surface.

Referring back to FIG. 1, after the surface of the micro-carrier has been modified, a solid-phase peptide synthesis step (step 204) is conducted, and the details of the solid-phase peptide synthesis step is described below. In this aspect of the present invention, the first amino acid that is being synthesized on the micro-carrier surface is tryptophan (TRP).

11. The carrier that comprises an aminated surface is placed inside a test tube, and argon gas is delivered into the test tube.

12. 228 mg of the Boc-Trp (tryptophan with a butyloxycarbonyl protecting group) powders is added to the test tube in step 11.

13. 2 ml of dichloromethane is then added to the test tube in step 12.

14. 118 μl liter of N,N′-diisopropylcarbondiimide and 1 ml of dimethyl formamide are added to the test tube in step 13.

15. The test tube in step 14 is shaken for 24 hours.

16. The carrier is removed from the test tube. The carrier is then washed for several times with dichloromethane (DCM) and dimethyl formamide (DMF), respectively. After a final rinse with dichloromethane, the carrier is vacuum dried.

After the completion of step 16, the amine group on the carrier is already attached to a Boc-Trp. The amine group of the amino acid on Trp is thus protected by a Boc group (protective group). The carboxyl end of Trp reacts with the amine group of the micro-carrier to form a peptide bond, and Trp is attached to the micro-carrier. The solid-phase peptide synthesis step is continued to remove the Boc group to expose the amine group of the amino acid on Trp. Removing the Boc group (protective group) is detailed below.

17. 1 ml of deionized water is added to 4 ml of tetrahydrofuran. The mixture is mixed evenly to form a reacting solution.

18. 4 ml of the reacting solution is removed to mix with 1 ml of the 97% H₂SO₄.

19. The reacting solution in step (18) is then added to a test tube with the carrier therein, wherein the carrier is attached to tryptophan with a Boc protecting group (Boc-Trp). The test tube is shaken for about 1 hour using an ultrasonic shaker.

20. The microcarrer in step 19 is then washed using deionized water and methanol, respectively. After a final wash with methanol, the wafer is vacuum dried.

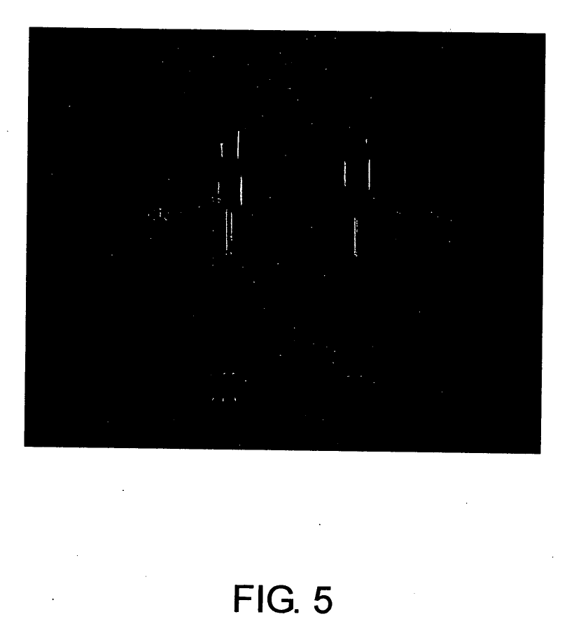

After completing the above process steps, a test is conducted to ensure the amine group of Trp is exposed. To test whether the amine group of Trp is exposed includes using the Ninhydrin assay. The detail of the analytical method of the Ninhydrin assay is the same as that being used to determine whether the micro-carrier surface has been modified to comprise an amine group. The analytical results are shown in FIG. 5. As shown in FIG. 5, a micro-carrier, having its surface covered only with a layer of silicon dioxide, is placed inside test tube 600, while a micro-carrier having a Trp group already attached to, is placed inside test tube 602. As shown in FIG. 5, test tube 600 exhibits a yellow color, while test tube 602 exhibits a violet color. Accordingly, the amine group of Trp on the micro-carrier in test tube 602 is exposed.

After completing step 20, the first amino acid, which is tryptophan (Trp), is attached to the micro carrier, wherein the amine group of Trp is exposed. After several repetitions of the synthesis steps and the protective group removal step (steps 11-20), a plural of amino acids can be sequentially attached to the micro-carrier to form a peptide that has a specific amino acid sequence.

Since the micro-carrier with a specific bar code (or number code) is synthesized with a peptide of a specific amino acid sequence, a correlation between the peptide with a specific amino acid sequence and the specific bar code (number code) can establish a coordinated information on a peptide and a bar code for the development of new peptide medicine.

The bipchip of the present invention is also applicable to the analysis of biological activity. Continuing to refer to FIG. 1, after step 204, step 206 is conducted in which a biological activity analysis is performed.

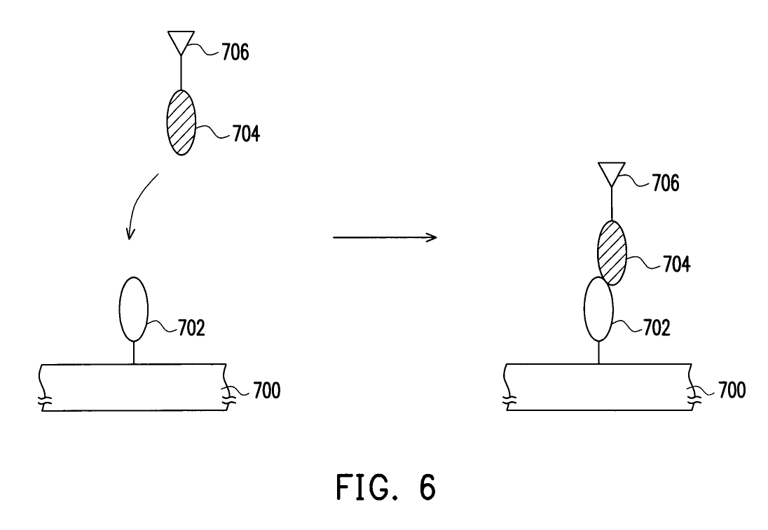

Referring to FIG. 6, a micro-carrier 700 with a peptide 702 of a specific amino acid sequence is placed in a reaction flask. A test-pending material 704 is added to the flask, wherein this test-pending material 704 is already labeled with a fluorescent dye 706.

If the test-pending material 704 and the peptide 702 with a specific amino acid sequence interacted, the test-pending material 704 would be coupled to the peptide 702 with the specific amino acid sequence. Since the test-pending material 704 is labeled with a fluorescent dye 706, the micro-carrier is also dyed.

Referring again to FIG. 1, after step 206, an image identification step (step 208) is conducted. The image identification step is detailed as follow. An identification system (for example, a microscope and an image analysis device) is used to identify the dyed micro-carrier in step 206. The identification method includes using the identification system to read the identification code (bar code or number code) on the micro-carrier. Since each identification code is corresponded to a peptide of a specific amino acid sequence, the test pending material is readily analyzed. In other words, the test-pending material that corresponds to that specific amino acid sequence is immediately identified.

Second Aspect of the Present Invention

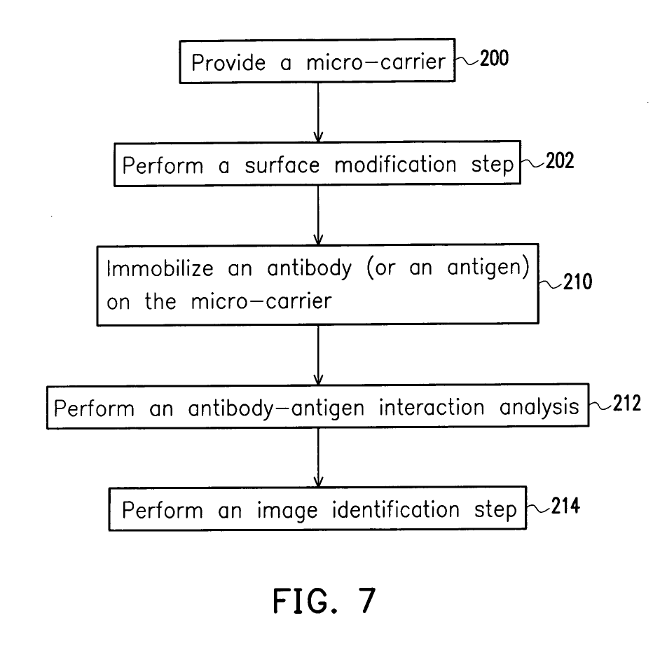

Referring to FIG. 7, FIG. 7 is a flow chart illustrating the fabrication method and the application of biochip according to another aspect of the present invention.

As shown in FIG. 7, a micro-carrier is provided (step 200), wherein the micro-carrier is already labeled with an identification code (for example, a bar code or a number code). Further, a material for forming the micro-carrier is, for example, polyethylene terephthalate (PET).

Thereafter, a surface modification step (step 202) is performed. In this aspect of the present invention, the surface modification step comprises coating a silicon dioxide layer on the surface of the micro-carrier, followed by modifying the silicon dioxide layer surface of the micro-carrier to an aminated surface. The surface modification process is already described in detail in the first embodiment and will not be reiterated here.

An antibody (or antigen) is then immobilized on the micro-carrier (step 210). The method to immobilize the antibody (or antigen) includes having the amine group on the aminated surface of the micro-carrier to react with the antibody (or antigen).

The antibody (or antigen) interaction analysis (step 212) is performed. Detail of the analysis is described below.

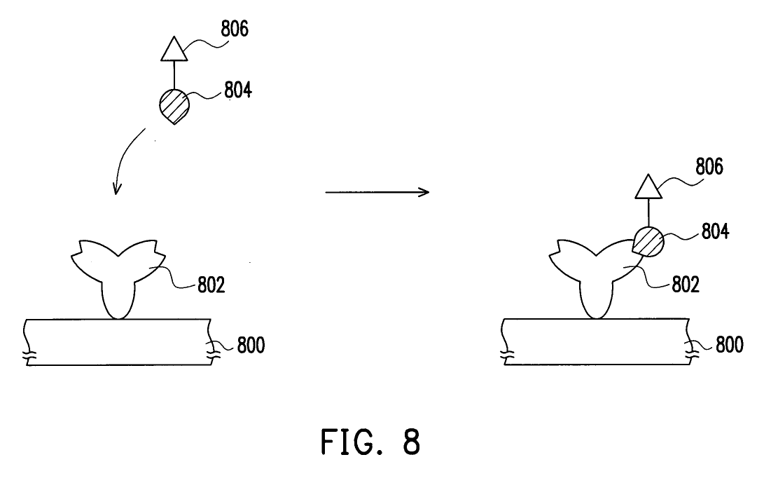

Referring to FIG. 8, micro-carrier 800, already having an antibody (or antigen) immobilized thereon, is placed in a reaction flask. A test-pending material 804 is also added to the reaction flask, wherein the test-pending material 804 is already labeled with a fluorescent dye 806.

Due to the unique and the specific binding characteristics of an antibody-antigen, the test-pending material is coupled to the antibody (or antigen) when the test-pending material 804 interacts with the antibody (or antigen) 802 that is immobilized on the micro-carrier. Since the test-pending material 804 is already labeled with a fluorescent material 806, the micro-carrier is dyed.

Referring to FIG. 7, after step 212, an image identification step (step 214) is conducted. The image identification step, which is detailed as follow, comprises using an identification system (for example, a microscope and an image analysis device) to identify the dyed micro-carrier in step 212. The identification method includes using the identification system to read the identification code (bar code or number code) on the micro-carrier. Since each identification code is correlated with a specific antibody (or antigen), the test-pending material that couples to the specific antibody (or antigen) is readily analyzed using the method of the present invention.

Accordingly, the present invention combines the synthetic chemistry for peptide and the bar-coded (or number-coded) micro-carrier to form a peptide or protein biochip. Further, an image identification technique is used to determine the interaction between a synthesized peptide (e.g. a ligand) and a receptor (e.g a film protein) for determining the biological activity. Moreover, an antibody (or antigen) immobilized on a bar-coded (number-coded) micro-carrier can be used to detect the interaction between an antibody and an antigen. One point that is worth noting is that using the micro-carrier that comprises a bar code (or a number code) to study the interaction between a ligand and a receptor or the interaction between an antibody-antigen, other identification procedures for confirming the biological molecule after the interaction can be precluded. Only an optical apparatus is required to read the bar code (or number code) to confirm the biological molecule.

In accordance to the fabrication method and the application of a biochip of the present invention, the microchip is labeled with a bar code or a number code. The sequence of the biological molecule or the test-pending molecule that corresponds to the antibody (or antigen) can be confirmed by using only an optical apparatus to directly read the bar code or the number code after the detection of the biological activity of the micro-carrier or the interaction between the antigen-antibody.

Further, the fabrication method and the application of the biochip of the present invention provides a speedy detection method for the biological activity of peptide or the interaction between antibody-antigen. The disadvantages of the prior art, for example, being time-consuming and labor intensive, thereby can be overcome.

Moreover, the fabrication method and the application of the biochip of the present invention can be applied directly on biological tissues to detect biological activity.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A fabrication method for a biochip, comprising:
providing a micro-carrier, wherein the micro-carrier is already labeled with an identification code;
performing a surface modification procedure to modify a surface of the micro-carrier to an aminated surface; and
performing a solid-phase peptide synthesis step to synthesize a peptide with a specific amino acid sequence on the aminated surface of the micro-carrier.

2. The method of claim 1, wherein the surface modification procedure comprises:
covering the surface of micro-carrier with a silicon dixoide layer; and
using 3-aminopropyltriethoxysilane to modify the silicon dioxide surface of the micro-carrier to the aminated surface.

3. The method of claim 1, wherein a material for forming the micro-carrier is a high molecular weight material.

4. The method of claim 1, wherein a material for forming the micro-carrier comprises polyethylene terephthalate (PET).

5. The method of claim 1, wherein the identification code on the micro-carrier is a bar code or a number code.

6. An application of the biochip as claimed in claim 1, comprising:
placing the micro-carrier with the peptide of the specific amino acid sequence in a reaction flask;
adding a test-pending material into the reaction flask, wherein the test-pending material is already labeled with a dye;
attaching the peptide of the specific amino acid sequence to the test-pending material to form a dyed microchip when there is an interaction between the test-pending material and the peptide with the specific amino acid sequence; and
using an identification system to identify the dyed micro-carrier and reading the identification code on the micro-carrier to analyze the test-pending material that corresponds to the peptide with the specific amino acid sequence.

7. The application of claim 6, wherein the identification system comprises a microscope and an image analysis device.

8. A fabrication method for a biochip, comprising:
providing a micro-carrier, wherein the micro-carrier is already labeled with an identification code;
performing a surface modification procedure to modify a property of a surface of the micro-carrier to an aminated surface; and
immobilizing an antibody (or antigen) on the aminated surface of the micro-carrier.

9. The method of claim 8, wherein the surface modification procedure further comprises:
covering a silicon dioxide layer on the surface of the micro-carrier; and
using 3-aminopropyltriethoxysilane to modify the silicon dioxide surface of the micro-carrier to the aminated surface.

10. The method of claim 8, wherein a material in forming the micro-carrier is a high molecular weight material.

11. The method of claim 8, wherein a material in forming the micro-carrier comprises polyethylene terephthalate (PET).

12. The method of claim 8, wherein the identification code on the micro-carrier includes a bar code or a number code.

13. An application of the biochip as claimed in claim 8, comprising:
placing the micro-carrier with the antibody (or antigen) immobilized thereon in a reaction flask;
adding a test-pending material into the reaction flask, wherein the test-pending material is labeled with a dye;
bonding the test-pending material to the antibody (or antigen) to form a dyed microchip when there is an interaction between the test-pending material and the antibody (or antigen); and
using an identification system to identify the dyed microchip and reading the identification code on the microchip to analyze the test-pending material that corresponds to the antibody (or antigen).

14. The application of claim 13, wherein if the antibody is immobilized on the micro-carrier, the test-pending material that interacts with the antibody is an antigen.

15. The method of claim 13, wherein if the antigen is immobilized on the micro-carrier, the test-pending material that interacts with the antigen is an antibody.

16. The method of claim 13, wherein the identification system comprises a microscope and an image analysis apparatus.
